(19) United States
(12) Patent Application Publication
(10) Pub. No.: US 2014/0350475 A1
(43) Pub. Date: Nov. 27, 2014

(54) MANUALLY OPERATED MEDICAL PUMP

(71) Applicant: SURGICAL APPS PTY LTD, Hornsby (AU)

(72) Inventors: Mark Lloyd Graham Bennett, Hornsby (AU); Roland Ian Stokes, Hornsby (AU); Leo Pinczewski, Hornsby (AU); Stephen Alan Scott, Hornsby (AU)

(21) Appl. No.: 14/368,712

(22) PCT Filed: Dec. 19, 2012
(86) PCT No.: PCT/AU2012/001557

§ 371 Date: Jun. 25, 2014

(30) Foreign Application Priority Data
Jan. 4, 2012 (AU) 2012900028
Jul. 23, 2012 (AU) 2012903117

(51) Int. Cl. A61M 5/142; A61M 5/168

(52) U.S. Cl. CPC A61M 5/1424; A61M 5/16881
USPC 604/185

(57) ABSTRACT

A hand operated medical pump is described including: at least one fluid inlet; at least one fluid outlet; a flexible pump bulb defining a pumping chamber which forms at least part of a pumping flow pathway; at least one directional valve which allows flow of fluid in a direction from the at least one inlet to the at least one outlet, but inhibits flow of fluid in a direction from the at least one outlet to the at least one inlet; and a bypass flow pathway from the at least one inlet to the at least one outlet which by-passes the at least one directional valve; and wherein the bypass flow pathway extends through the pump bulb.

### TECHNICAL FIELD

The invention relates to pumps and more particularly to hand operated pumps for use in medical applications. The invention has been developed primarily for use as in arthroscopic surgery and will be described hereinafter with reference to this application. However, it will be appreciated that the invention is not limited to this particular use. Embodiments of the invention are also suitable for pumping blood and other fluids in medical applications.

### BACKGROUND TO THE INVENTION

During arthroscopic surgery a steady flow of sterile saline fluid is applied to a region being operated on to clear the operative field of blood and bone debris. From time to time, it is desirable to apply a large volume bolus of fluid to the operative field to, for example, expand the joint being operated on, or to clear stubborn debris.

An arrangement described in WO2004/023987 utilises two manually operated pump bodies. Both pumps can supply a slow steady flow of fluid held in overhead multiple fluid reservoirs by the action of gravity. However, in order to provide a high volume flow it is necessary to pump each pump sequentially. This operation requires two free hands and therefore cannot easily be carried out by a surgeon during an operation.

An arrangement described in U.S. Pat. No. 5,507,707 utilises one manually operated pump body. A short length of tubing bypasses the pump body to provide a continuous trickle of flow which can be augmented by operating the pump. However, in order to operate the pump, the surgeon must carefully insert his fingers between the bypass tubing and the pump body and take care not to disrupt the bypass tubing. The surgeon therefore must divert their eyes from the operative filed in order to operate the pump and must take care when operating the pump not to accidentally disrupt the bypass tubing.

There remains a need for improved pumping devices which are able to provide both a continuous steady flow of fluid and can provide an increased flow of fluid on demand by manual operation of the pumping device.

### SUMMARY OF THE INVENTION

In a first aspect the present invention provides a hand operated medical pump including: at least one fluid inlet; at least one fluid outlet; a flexible pump bulb defining a pumping chamber which forms at least part of a pumping flow pathway; at least one directional valve which allows flow of fluid in a direction from the at least one inlet to the at least one outlet, but inhibits flow of fluid in a direction from the at least one outlet to the at least one inlet; and a bypass flow pathway from the at least one inlet to the at least one outlet which bypasses the at least one directional valve; and wherein the bypass flow pathway extends through the pump bulb.

At least a portion of the bypass flow pathway may be formed by a tube which is at least partially located within the pump bulb.

The pump may include two fluid inlets, one of which is associated with the bypass flow pathway.

The pump may include two directional valves, each located respectively upstream and downstream of the pumping chamber.

The pumping flow pathway and the bypass flow pathway may discharge into a common output chamber.

The pump bulb may be transparent.

The at least one directional valve may include a flap formation.

The flap formation may be embodied in a flexible washer.

### BRIEF DESCRIPTION OF THE DRAWINGS

Embodiments of the present invention will now be described, by way of example only, with reference to the accompanying drawings, in which:

FIG. 1 is a side view of a pump;

FIG. 2 is cross sectional plan view of the pump depicted in FIG. 1, taken along line A-A;

FIG. 3 is an enlarged partial cross sectional plan view of the pump depicted in FIG. 1, taken along line A-A;

FIG. 4 is a cross sectional view of an input hub and insert;

FIG. 5 is a cross sectional view of an output hub and insert;

FIG. 6 is an end view, partially sectioned illustrating an input hub and valve arrangement; and

FIG. 7 is a second embodiment of an output hub and insert illustrating a concentric discharge.

### DETAILED DESCRIPTION OF THE PREFERRED EMBODIMENT

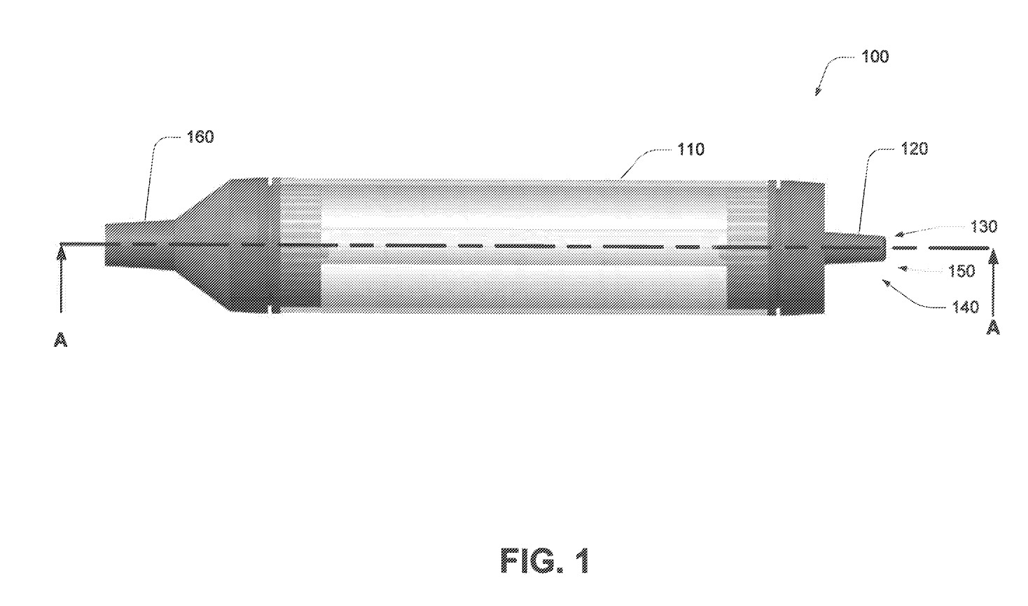

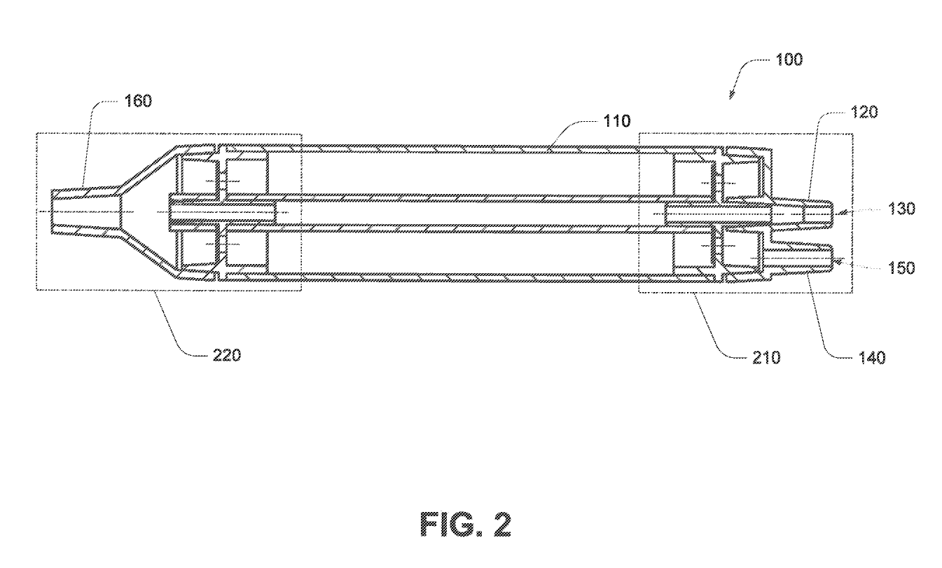

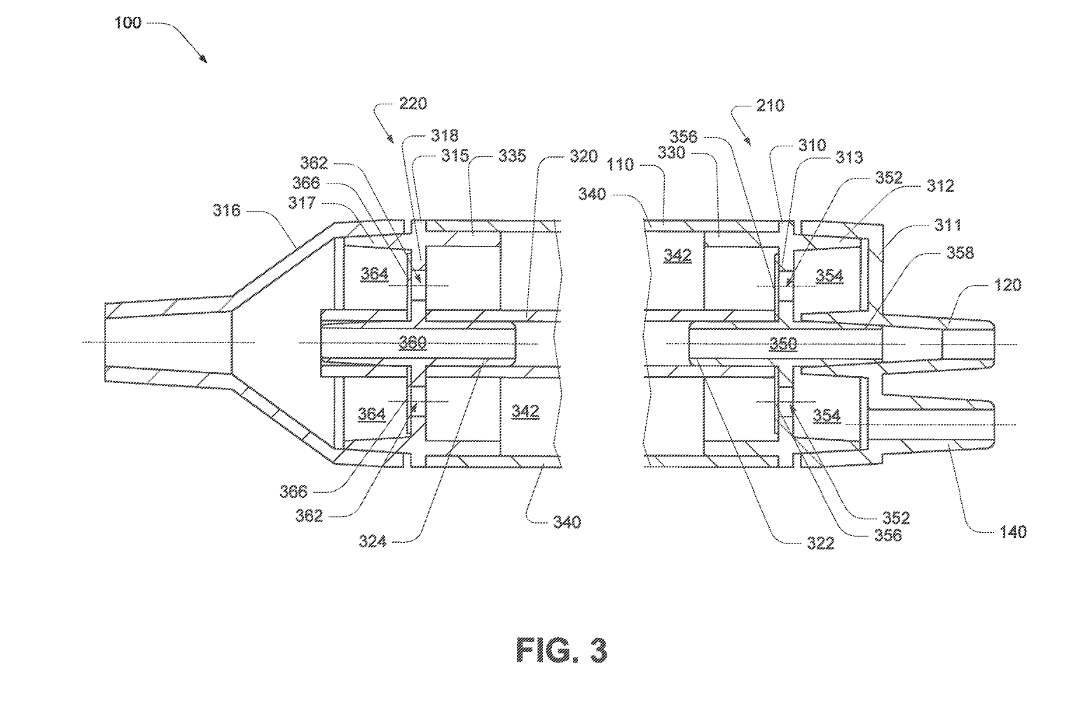

Referring to FIG. 1 through FIG. 3, a hand operated pump 100 comprises a generally cylindrical body 110 having two inlets or nipples and one outlet nozzle. A bypass inlet 120 receives a first fluid supply 130 and a pumping inlet 140 (as shown in FIG. 2 and FIG. 3) receives a second fluid supply 150. The pump 100 discharges through a single discharge nozzle 160.

The fluid supplies are typically provided in the form of suspended bags of fluid which are connected to the inlets by way of PVC tubes in a conventional manner. The fluid may be a saline solution, blood, or other fluid or combination of fluids which may vary according to the particular application that the pump is to be used for.

Inlets 120, 140 can attach to tubes of at least two sizes depending upon the application at hand. A larger diameter tube fits about the outside of inlets 120, 140. A smaller diameter tube can be push fitted into inlets 120, 140.

Referring to FIG. 3, the body of the pump 100 is built around two similar hubs, being an inlet hub 310 and a discharge hub 315. Each hub, 310 and 315, has a generally cylindrical collar for retaining an end cap. The inlet cap 311 is retained, attached to and sealed against the inlet hub collar 312. The discharge cap 316 is attached to and sealed against the discharge hub collar 317.

The two hubs, 310 and 315, are interconnected in two ways. A soft PVC (or rigid) bypass tube 320 extends between the two hubs by affixing to a first inwardly directed bypass nozzle 322 associated with the inlet hub 310 and a second bypass nozzle 324 that extends inwardly from the discharge hub 315. The bypass tube 320 may be fitted to the first nozzle 322 and second nozzle 324, for example by way of any one of the following: a frictional fit, an interference fit, a weldment or an adhesive etc.

Each hub, 310 and 315, also has an inward facing, axially extending and generally cylindrical sleeve 330 and 335 respectively. The sleeves, 330 and 335, support opposites ends of a pump jacket (or bulb) 340. In this example embodiment, the pump jacket (or bulb) is a flexible cylindrical PVC pump jacket. The cylindrical jacket 340 distorts when squeezed to alter (by reducing) the internal volume of the pumping chamber 342. The pumping chamber 342 is generally cylindrical. When released the jacket 340 returns to its original shape.

The inlet hub 310 has a central circular web 313 in which is formed one or more through openings 352 located around the central bypass channel 350. The openings 352 lead from the intake chamber 354 to the pumping chamber 342. Fluid can be manually pumped through a pumping flow path of pump device 100. Manually pumped fluid enters the inlet nozzle 140 and passes through the intake chamber 354. From the intake chamber 354, fluid flows through the inlet ports 352 and into the pumping chamber 342. Back flow from the pumping chamber 342 into the intake chamber 354 is prevented by one or more flap type check valves 356 as will be explained with reference to FIGS. 4-7.

Fluid in the pumping chamber 342 is discharged through the one or more discharge ports 362 formed in the central membrane of the discharge hub 315. The discharge ports 362 lead into a discharge chamber 364. The discharge chamber 364 is defined by circular web 318 of the discharge hub and the discharge end cap 316. Back flow from the discharge chamber 364 is prevented by one or more check valves 366.

Fluid flow through the pump's bypass flow path proceeds from the bypass inlet or nipple 120, through a second bypass inlet nipple 358 that fits into it and is associated with the inlet hub 310. Flow proceeds through the nozzle's bypass channel 350, via the bypass tube 320, flowing through the central opening of the discharge hub's bypass channel 360 and into the discharge chamber 364. Fluid from the pumping inlet 140 proceeds into the discharge chamber 364 when the jacket 340 is squeezed by the operator. The manually pumped and the bypass flows combine in the discharge chamber 364 and exit through the single discharge nozzle 160.

In preferred embodiments, the generally cylindrical jacket 340 is at least partially transparent so that the internal contents of the pump 100 may be viewed by the operator. The bypass tube 320 may also be transparent.

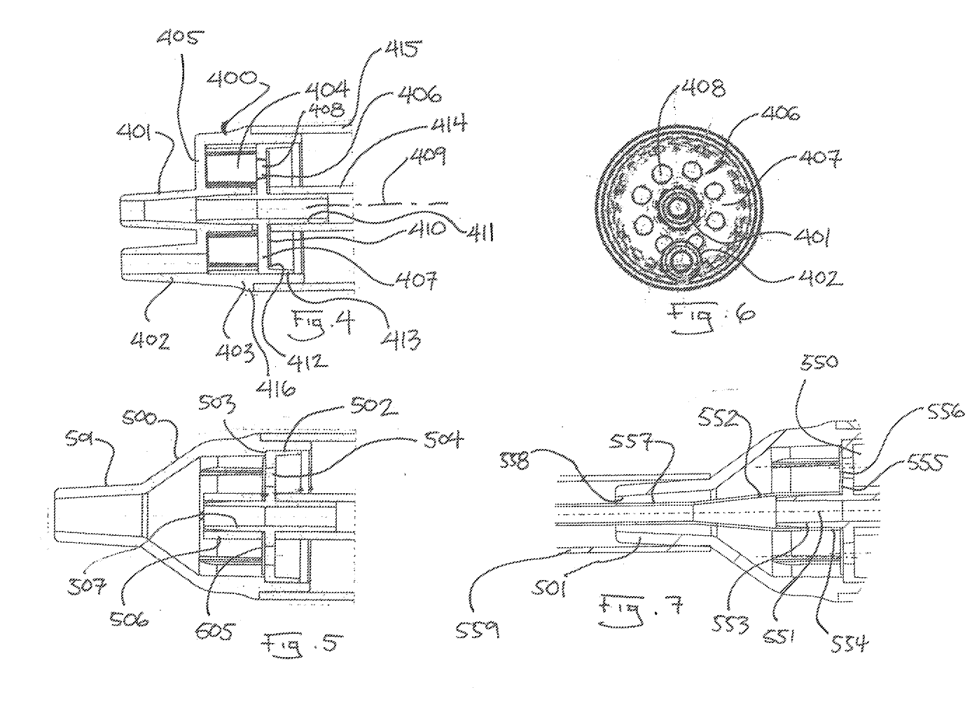

Further embodiments of the invention are depicted in FIGS. 4-7. As shown in FIG. 4, an input hub 400 comprises an external bypass inlet or nipple 401 and a pumping inlet or nipple 402. The hub 400 has a cylindrical outer wall or sleeve 403 that defines, in part, an intake chamber 404. The intake chamber 404 is also defined by the end wall 405 of the hub and the external face of an intake insert 406. As suggested by FIGS. 4 and 6, the intake insert 406 comprises a generally circular web 407 in which is formed one or more intake openings 408. In this example, the openings 408 are arranged in a circular array of equally spaced round openings, all generally equidistant from the longitudinal centre line of the pump 409. In some embodiments, the openings 408 are tapered from a larger diameter toward the intake to a smaller diameter toward the output side of the pump. In this example, the insert serves as a check valve. A thin silicone washer 410 has a central opening that fits over and clears the inwardly directed bypass nozzle 411. The outer diameter 412 of the silicone flapper or valve element 410 fits within the insert's cylindrical shoulder 413. The valve element 410 is retained about its central opening by the bypass tube 414. Accordingly, pressure in the intake chamber 404 is relieved by fluid passing through the openings 408 and past the valve element 410. When the pressure equilibrates, the valve element 410 returns to its initial shape and position as shown in FIG. 4. The pump jacket 415 is attached to the exterior of the hub 400 and its axial movement is limited by a shoulder 416 formed on the outer surface of the hub.

As shown in FIG. 5, the discharge hub 500 is constructed in accordance with the teachings related to FIGS. 4 and 6. In this example, the output hub 500 forms a single, tapered, discharge nozzle 501. A discharge insert 502 fits within the discharge hub and abuts an internal shoulder 503. The insert 502 has an array of discharge openings 504. A silicone washer or valve element 505 prevents backflow and is retained by a short tubular formation 506 that fits over the tapered discharge outlet 507 formed integrally with the insert 502. The discharge outlet 507 may be provided with a shoulder or ridge which serves as a stop to locate and retain the tubular formation 502 on the discharge outlet. The tubular formation may be provided with an internal groove which cooperates with the ridge. This ensures that silicone washer 505 is not “pinched” by tubular formation 502 to ensure satisfactory operation of the one way valve arrangement.

As shown in FIG. 7, an alternate embodiment discharges the bypass flow separately from the flow through the pumping chamber 550. Flow through 20 the insert's bypass channel 551 is captured by a preferably tapered conduit 552 that fits over the bypass insert output nozzle 553. The base end 554 of the conduit 552 serves to retain or capture the inside diameter 555 of the silicone or other polymeric valve element 556 and thus replaces the function of the tubular formation 506 shown in FIG. 5. The conduit 552 tapers to a smaller diameter 557 that can exit the discharge hub's output nozzle 501 with enough clearance 558 between the outside diameter of the conduit 557 and inside diameter of the nozzle 501 to accommodate the flow from the pumping chamber 550. Accordingly, the pump output comprises concentric flow lines being the larger one 559 for carrying the output of the pumping chamber and the smaller one, within it and preferably concentrically for 557 carrying the bypass flow. The purpose of the arrangement of concentric or otherwise separate outputs is to prevent backflow up the bypass line.

It will be appreciated from the above teachings that the thin, low threshold flexible silicone check valve elements allow flow through the pump even when the pump is not being manually or otherwise activated. The design incorporates no valve elements in the bypass flow path which allows a continuous flow of liquid under the action of gravity from the supply of fluid.

In some embodiments, the two inlets may be consolidated into a single inlet which opens into an inlet chamber and the pumping flow pathway and the bypass flow pathway share this common inlet chamber.

Embodiments of the invention therefore provide a manually operated pump that provides both manually pressurized fluid as well as a continuous flow of fluid through a single pumping device that can be easily operated with one hand. The bypass flow path is protected within the pump bulb and cannot be accidentally disrupted or dislodged during manual pumping operations.

It will be appreciated that an embodiment of the invention can consist essentially of features disclosed herein. Alternatively, an embodiment of the invention can consist of features disclosed herein. The invention illustratively disclosed herein suitably may be practiced in the absence of any element which is not specifically disclosed herein.

Reference throughout this specification to “one embodiment” or “an embodiment” means that a particular feature, structure or characteristic described in connection with the embodiment is included in at least one embodiment of the present invention. Thus, appearances of the phrases “in 15 one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, unless otherwise specified the use of terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader, or with reference to the orientation of the structure during nominal use, as appropriate. Similarly, the terms “inwardly” and “outwardly” generally refer to the orientation of a surface relative to its axis of elongation, or axis of rotation, as appropriate.

Similarly it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Any reference to prior art contained herein is not to be taken as an admission that the information is common general knowledge, unless otherwise indicated.

Finally, it is to be appreciated that various alterations or additions may be made to the parts previously described without departing from the spirit or ambit of the present invention.

1. A hand operated medical pump including:
at least one fluid inlet;
at least one fluid outlet;
a flexible pump bulb defining a pumping chamber which forms at least part of a pumping flow pathway;
at least one directional valve which allows flow of fluid in a direction from the at least one inlet to the at least one outlet, but inhibits flow of fluid in a direction from the at least one outlet to the at least one inlet; and
a bypass flow pathway from the at least one inlet to the at least one outlet which bypasses the at least one directional valve;
and wherein the bypass flow pathway extends through the pump bulb.

2. A pump according to claim 1 wherein at least a portion of the bypass flow pathway is formed by a tube which is at least partially located within the pump bulb.

3. A pump according to claim 1 which includes two fluid inlets, one of which is associated with the bypass flow pathway.

4. A pump according to claim 1 which includes two directional valves, each located respectively upstream and downstream of the pumping chamber.

5. A pump according to claim 1 wherein the pumping flow pathway and the bypass flow pathway discharge into a common output chamber.

6. A pump according to claim 1 wherein the pump bulb is transparent.

7. A pump according to claim 1 wherein the at least one directional valve includes a flap formation.

8. A pump according to claim 7 wherein the flap formation is embodied in a flexible washer.
